(19) United States
(12) Patent Application Publication
(10) Pub. No.: US 2007/0105827 A1
(43) Pub. Date: May 10, 2007

(54) Estrogen receptor modulators

(76) Inventors: Timothy A. Blizzard, Princeton, NJ (US); Candido Gude, Staten Island, NY (US); Jerry D. Morgan II, Woodbridge, NJ (US)

Correspondence Address: MERCK AND CO., INC, P O BOX 2000, RAHWAY, NJ, 07065-0907, US

(21) Appl. No.: 10/579,652

(22) PCT Filed: Nov. 19, 2004
(86) PCT No.: PCT/US04/39050

§ 371 Date: May 18, 2006

Related U.S. Application Data
(60) Provisional application No. 60/524,580, filed on Nov. 24, 2003.

(51) Int. Cl. A61K 31/56

(52) U.S. Cl. 514/169

(57) ABSTRACT

The present invention relates to compounds and derivatives thereof, their synthesis, and their use as estrogen receptor modulators. The compounds of the instant invention are ligands for estrogen receptors and as such may be useful for treatment or pre-vention of a variety of conditions related to estrogen functioning including: bone loss, bone fracturs, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, inflammation, inflammatory bowel disease, sexual dysfunction, hypertension, retinal degeneration and cancer, in particular of the breast, uterus and prostate.

### BACKGROUND OF THE INVENTION

Naturally occurring and synthetic estrogens have broad therapeutic utility, including: relief of menopausal symptoms, treatment of acne, treatment of dysmenorrhea and dysfunctional uterine bleeding, treatment of osteoporosis, treatment of hirsutism, treatment of prostatic cancer, treatment of hot flashes and prevention of cardiovascular disease. Because estrogen is very therapeutically valuable, there has been great interest in discovering compounds that mimic estrogen-like behavior in estrogen responsive tissues.

The estrogen receptor has been found to have two forms: ERα and ERβ. Ligands bind differently to these two forms, and each form has a different tissue specificity to binding ligands. Thus, it is possible to have compounds that are selective for ERα or ERβ, and therefore confer a degree of tissue specificity to a particular ligand.

What is needed in the art are compounds that can produce the same positive responses as estrogen replacement therapy without the negative side effects. Also needed are estrogen-like compounds that exert selective effects on different tissues of the body.

The compounds of the instant invention are ligands for estrogen receptors and as such may be useful for treatment or prevention of a variety of conditions related to estrogen functioning including: bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

### SUMMARY OF THE INVENTION

The present invention relates to methods of treating or preventing a variety of conditions related to estrogen functioning. One embodiment of the present invention is illustrated by treating or preventing estrogen related disorders with a compound of Formula I, and the pharmaceutically acceptable salts and stereoisomers thereof:

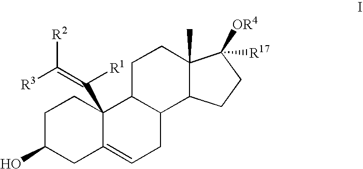

### DETAILED DESCRIPTION OF THE INVENTION

The present invention relates to methods of treating or preventing a variety of conditions related to estrogen functioning. One embodiment of the present invention is illustrated by treating or preventing disease with a compound of Formula I, and the pharmaceutically acceptable salts and stereoisomers thereof: 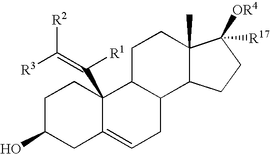
wherein R¹ is hydrogen, halo, C₍₁₋₃₎ alkyl, CO₂C₍₁₋₃₎alkyl, or cyano;
R² is hydrogen, halo, C₍₁₋₃₎ alkyl, CO₂C₍₁₋₃₎alkyl, or cyano;
R³ is hydrogen, halo, C₍₁₋₃₎ alkyl, CO₂C₍₁₋₃₎alkyl, or cyano;
R⁴ is hydrogen, or C₍₁₋₃₎ alkyl;
R¹⁷ is hydrogen, C₍₁₋₅₎ alkyl, C₍₁₋₅₎ acyl, C₍₂₋₅₎ alkenyl, or C₍₂₋₅₎ alkynyl;
   and the pharmaceutically acceptable salts and stereoisomers thereof;
   wherein said disease is: bone loss, bone fractures, osteoporosis, metastaic bone disease, Paget's disease, periodontal disease, cartilage degeneration, endometriosis, uterine fibroid disease, hot flashes, cardiovascular disease, impairment of cognitive functioning, cerebral degenerative disorders, restenosis, gynecomastia, vascular smooth muscle cell proliferation, obesity, incontinence, anxiety, depression, perimenopausal depression, postpartum depression, premenstrual syndrome, manic depression, anxiety, dementia, obsessive compulsive behavior, attention deficit disorder, sleep disorders, irritability, impulsivity, anger management, multiple sclerosis, Parkinson's disease, inflammation, inflammatory bowel disease, sexual dysfunction, hypertension, retinal degeneration or an estrogen dependent cancer.

In an embodiment of the invention,
R¹ is hydrogen, halo, methyl or cyano;
R² is hydrogen, halo, methyl or cyano;
R³ is hydrogen, halo, methyl or cyano;
R⁴ is hydrogen, or C₍₁₋₃₎ alkyl;
R¹⁷ is hydrogen, C₍₁₋₃₎ alkyl, C₍₂₋₃₎ acyl, C₍₂₋₃₎ alkenyl, or C₍₁₋₃₎ alkynyl;
   and the pharmaceutically acceptable salts and stereoisomers thereof.

The presnt invention also relates to a pharmaceutical composition comprising a compound of the formula 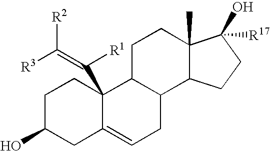
wherein R1 is hydrogen, halo, C₍₁₋₃₎ alkyl, CO₂C₍₁₋₃₎alkyl, or cyano;
R2 is hydrogen, halo, C₍₁₋₃₎ alkyl, CO₂C₍₁₋₃₎alkyl, or cyano;
R3 is hydrogen, halo, C₍₁₋₃₎ alkyl, CO₂C₍₁₋₃₎alkyl, or cyano;
R17 is hydrogen, C₍₁₋₅₎ alkyl, C₍₁₋₅₎ acyl, C₍₂₋₅₎ alkenyl, or C₍₁₋₅₎ alkynyl;
   and the pharmaceutically acceptable salts and stereoisomers thereof;
   and another agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; or a selective serotonin reuptake inhibitor; an aromatase inhibitor, or a pharmaceutically acceptable salt or mixture thereof.

The present invention also related to a compound of formula II: 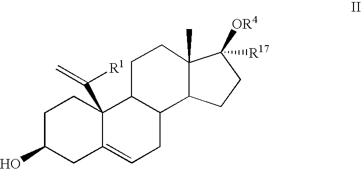
wherein R¹ is hydrogen, halo, C₍₁₋₃₎ alkyl or cyano;
R⁴ is hydrogen, or C₍₁₋₃₎ alkyl;
R¹⁷ is hydrogen, C₍₁₋₅₎ alkyl, C₍₁₋₂₎ alkenyl, or C₍₂₋₅₎ alkynyl;
   with the proviso that R¹ and R⁴ are not both hydrogen and the pharmaceutically acceptable salts and stereoisomers thereof.

Non-limiting examples of the present invention include, but are not limited to:
19-nor-10β-vinyl-3β,17β-androst-5-ene diol (R¹═R²═R³═R⁴═R¹⁷═H);
19-nor-10β-vinyl-3β-hydroxy-17β-methoxy-androst-5-ene (R¹═R²═R³═R¹⁷═H; R⁴═CH₃);
17α-ethynyl-19-nor-10β-vinyl-3β,17β-androst-5-ene diol (R¹═R²═R³═R⁴═H; R¹⁷═CCH);
17α-vinyl-19-nor-10β-vinyl-3β,17β-androst-5-ene diol (R¹═R²═R³═R⁴═H; R¹⁷═CHCH₂);
17α-methyl-19-nor-10β-vinyl-3β,17β-androst-5-ene diol (R¹═R²═R³═R⁴═H; R¹⁷═CH₃);
19-nor-10β-(1-methyl-vinyl)-3β,17β-androst-5-ene diol (R¹═CH₃; R²═R³═R⁴═R¹⁷═H);
19-nor-10β-(cis-2-methyl-vinyl)-3β,17β-androst-5-ene diol (R¹═R²═H; R³═CH₃; R⁴═R¹⁷═H);
19-nor-10β-(trans-2-methyl-vinyl)-3β,17β-androst-5-ene diol (R¹═H; R²═CH₃; R³═R⁴═R¹⁷═H);
19-nor-10β-(1-ethyl-vinyl)-3β,17β-androst-5-ene diol (R¹═CH₂CH₃; R²═R³═R⁴═R¹⁷═H);
19-nor-10β-(cis-2-ethyl-vinyl)-3β,17β-androst-5-ene diol (R^{1═R}²═H; R³═CH₂CH₃; R⁴═R¹⁷═H);
19-nor-10β-(trans-2-ethyl-vinyl)-3β,17β-androst-5-ene diol (R¹═H; R²═CH₂CH₃; R³═R⁴═R¹⁷═H)
19-nor-10β-(1-chloro-vinyl)-3β,17β-androst-5-ene diol (R¹═Cl; R²═R³═R⁴═R¹⁷═H);
19-nor-10β-(cis-2-chloro-vinyl)-3β,17β-androst-5-ene diol (R¹═R²═H; R³═Cl; R⁴═R¹⁷═H);
19-nor-10β-(trans-2-chloro-vinyl)-3β,17β-androst-5-ene diol (R¹═H; R²═Cl; R³═R⁴═R¹⁷═H);
19-nor-10β-(1-fluoro-vinyl)-3β,17β-androst-5-ene diol (R¹═F; R²═R³═R⁴═R¹⁷═H);
19-nor-10β-(cis-2-fluoro-vinyl)-3β,17β-androst-5-ene diol (R¹═R²═H; R³═F; R⁴═R¹⁷═H);
19-nor-10β-(trans-2-fluoro-vinyl)-3β,17β-androst-5-ene diol (R¹═H; R²═F; R³═R⁴═R¹⁷═H);
19-nor-10β-(2,2-difluoro-vinyl)-3β,17β-androst-5-ene diol (R¹═H; R²═R³═F; R⁴═R¹⁷═H);
19-nor-10β-(trifluorovinyl)-3β,17β-androst-5-ene diol (R¹═R²═R³═F; R⁴═R¹⁷═H);
17α-ethynyl-19-nor-10β-trifluorovinyl-3β,17β-androst-5-ene diol (R¹═R²═R³═F; R⁴═R¹⁷═CCH);
   and the pharmaceutically acceptable salts thereof.

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application. The present invention also relates to methods for making the pharmaceutical compositions of the present invention. The present invention is also related to processes and intermediates useful for making the compounds and pharmaceutical compositions of the present invention. These and other aspects of the invention will be apparent from the teachings contained herein.

Utilities

The compounds of the present invention are selective modulators of estrogen receptors and are therefore useful to treat or prevent a variety of diseases and conditions related to estrogen receptor functioning in mammals, preferably humans.

A variety of diseases and conditions related to estrogen receptor functioning includes, but is not limited to, bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, perimenopausal depression, post-partum depression, premenstrual syndrome, manic depression, anxiety, dementia, obsessive compulsive behavior, attention deficit disorder, sleep disorders, irritability, impulsivity, anger management, multiple sclerosis and Parkinson's disease, inflammation, inflammatory bowel disease, sexual dysfunction, hypertension, retinal degeneration and cancer, in particular of the breast, uterus and prostate. In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

The present invention also relates to methods for eliciting an estrogen receptor modulating effect in a mammal in need thereof by administering the compounds and pharmaceutical compositions of the present invention.

The present invention also relates to methods for eliciting an estrogen receptor antagonizing effect in a mammal in need thereof by administering the compounds and pharmaceutical compositions of the present invention. The estrogen receptor antagonizing effect can be either an ERα antagonizing effect, an ERβ antagonizing effect or a mixed ERα and ERβ antagonizing effect.

The present invention also relates to methods for eliciting an estrogen receptor agonizing effect in a mammal in need thereof by administering the compounds and pharmaceutical compositions of the present invention. The estrogen receptor agonizing effect can be either an ERα agonizing effect, an ERβ agonizing effect or a mixed ERα and ERβ agonizing effect. A preferred method of the present invention is eliciting an ERβ agonizing effect.

The present invention also relates to methods for treating or preventing disorders related to estrogen functioning, bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomnastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate in a mammal in need thereof by administering the compounds and pharmaceutical compositions of the present invention. Exemplifying the invention is a method of treating or preventing depression. Exemplifying the invention is a method of treating or preventing anxiety. Exemplifying the invention is a method of treating or preventing hot flashes. Exemplifying the invention is a method of treating or preventing cancer. Exemplifying the invention is a method of treating or preventing cardiovascular disease.

An embodiment of the invention is a method for treating or preventing cancer, especially of the breast, uterus or prostate, in a mammal in need thereof by administering the compounds and pharmaceutical compositions of the present invention. The utility of SERMs for the treatment of breast, uterine or prostate cancer is known in the literature, see T. J. Powles, “Breast cancer prevention,” Oncologist 2002; 7(1):604; Park, W. C. and Jordan, V. C., “Selective estrogen receptor modulators (SERMS) and their roles in breast cancer prevention.” Trends Mol Med. 2002 February;8(2):82-8; Wolff, A. C. et al., “Use of SERMs for the adjuvant therapy of early-stage breast cancer,” Ann N Y Acad Sci. 2001 December;949:80-8; Steiner, M. S-et al., “Selective estrogen receptor modulators for the chemoprevention of prostate cancer,” Urology 2001 April; 57(4 Suppl 1):68-72.

Another embodiment of the invention is a method of treating or preventing metastatic bone disease in a mammal in need thereof by administering to the mammal a therapeutically effective amount of any of the compounds or pharmaceutical compositions described above. The utility of SERMS in the treatment of metastatic bone disease is known in the literature, see, Campisi, C. et al., “Complete resoultion of breast cancer bone metastasis through the use of beta-interferon and tamoxifen,” Eur J Gynaecol Oncol 1993;14(6):479-83.

Another embodiment of the invention is a method of treating or preventing gynecornastia in a mammal in need thereof by administering to the mammal a therapeutically effective amount of any of the compounds or pharmaceutical compositions described above. The utility of SERMS in the treatment of gynecomastia is known in the literature, see, Ribeiro, G. and Swindell R., “Adjuvant tamoxifen for male breast cancer.” Br J Cancer 1992;65:252-254; Donegan, W., “Cancer of the Male Breast,” JGSM Vol. 3, Issue 4, 2000.

Another embodiment of the invention is a method of treating or preventing post-menopausal osteoporosis, glucocorticoid osteoporosis, hypercalcemia of malignancy, bone loss and bone fractures in a mammal in need thereof by administering to the mammal a therapeutically effective amount of any of the compounds or pharmaceutical compositions described above. The utility of SERMs to treat or prevent osteoporosis, hypercalcemia of malignancy, bone loss or bone fractures is known in the literature, see Jordan, V. C. et al., “Selective estrogen receptor modulation and reduction in risk of breast cancer, osteoporosis and coronary heart disease,” Natl Cancer Inst 2001 October; 93(19):1449-57; Bjamason, N H et al., “Six and twelve month changes in bone turnover are realted to reduction in vertebral fracture risk during 3 years of raloxifene treatment in postemenopausal osteoporosis,” Osteoporosis Int 2001; 12(11):922-3; Fentiman I. S., “Tamoxifen protects against steroid-induced bone loss,” Eur J Cancer 28:684-685 (1992); Rodan, G. A. et al., “Therapeutic Approaches to Bone Diseases,” Science Vol 289, 1 September 2000.

Another embodiment of the invention is a method of treating of preventing periodontal disease or tooth loss in a mammal in need thereof by administering to the mammal a therapeutically effective amount of any of the compounds or pharmaceutical compositions described above. The use of SERMs to treat periodontal disease or tooth loss in a mammal is known in the literature, see Rodan, G. A. et al., “Therapeutic Approaches to Bone Diseases,” Science Vol 289, 1 September 2000 pp. 1508-14.

Another embodiment of the invention is a method of treating of preventing Paget's disease in a mammal in need thereof by administering to the mammal a therapeutically effective amount of any of the compounds or pharmaceutical compositions described above. The use of SERMs to treat Paget's disease in a mammal is known in the literature, see Rodan, G. A. et al., “Therapeutic Approaches to Bone Diseases,” Science Vol 289, 1 September 2000 pp. 1508-14.

Another embodiment of the invention is a method of treating or preventing uterine fibroid disease in a mammal in need thereof by administering to the mammal a therapeutically effective amount of any of the compounds or pharmaceutical compositions described above. The use of SERMS to treat uterine fibroids, or uterine leiomyomas, is known in the literature, see Palomba, S., et al, “Effects of raloxifene treatment on uterine leiomyomas in postmenopausal women,” Fertil Steril. 2001 July;76(1):38-43.

Another embodiment of the invention is a method of treating or preventing obesity in a mammal in need thereof by administering to the mammal a therapeutically effective amount of any of the compounds or pharmaceutical compositions described above. The use of SERMs to treat obesity is known in the literature, see Picard, F. et al., “Effects of the estrogen antagonist EM-652.HCl on energy balance and lipid metabolism in ovariectomized rats,” Int J Obes Relat Metab Disord. 2000 Jul. 24(7):830-40.

Another embodiment of the invention is a method of treating or preventing cartilage degeneration, rheumatoid arthritis or osteoarthritis in a mammal in need thereof by administering to the mammal a therapeutically effective amount of any of the compounds or pharmaceutical compositions described above. The use of SERMs to treat cartilage degeneration, rheumatoid arthritis or osteoarthritis is known in the literature, see Badger, A. M. et aZ., “Idoxifene, a novel selective estrogen receptor modulator, is effective in a rat model of adjuvant-induced arthritis.” J Pharmacol Exp Ther. 1999 December;291(3): 1380-6.

Another embodiment of the invention is a method of treating or preventing endometriosis in a mammal in need thereof by administering to the mammal a therapeutically effective amount of any of the compounds or pharmaceutical compositions described above. The use of SERMs to treat endometriosis is known in the art, see Steven R. Goldstein, “The Effect of SERMs on the Endometriur,” Annals of the New York Academy of Sciences 949:237-242 (2001).

Another embodiment of the invention is a method of treating or preventing urinary incontinence in a mammal in need thereof by administering to the mammal a therapeutically effective amount of any of the compounds or pharmaceutical compositions described above. The use of SERMs to treat urinary incontinence is known in the art, see, Goldstein, S. R., “Raloxifene effect on frequency of surgery for pelvic floor relaxation,” Obstet Gynecol. 2001 July; 98(1):91-6.

Another embodiment of the invention is a method of treating or preventing cardiovascular disease, restenosis, lowering levels of LDL cholesterol and inhibiting vascular smooth muscle cell proliferation in a mammal in need thereof by administering to the mammal a therapeutically effective amount of any of the compounds or pharmaceutical compositions described above. Estrogen appears to have an effect on the biosynthesis of cholesterol and cardiovascular health. Statistically, the rate of occurrence of cardiovascular disease is roughly equal in postmenopausal women and men; however, premenopausal women have a much lower incidence of cardiovascular disease than men. Because postmenopausal women are estrogen deficient, it is believed that estrogen plays a beneficial role in preventing cardiovascular disease. The mechanism is not well understood, but evidence indicates that estrogen can upregulate the low density lipid (LDL) cholesterol receptors in the liver to remove excess cholesterol. The utility of SERMs in treating or preventing cardiovascular disease, restenosis, lowering levels of LDL cholesterol and inhibiting vascular smooth muscle cell proliferation is known in the art, see Nuttall, M E et al., “Idoxifene: a novel selective estrogen receptor modulator prevents bone loss and lowers cholesterol levels in ovariectomized rats and decrease& uterine weight in intact rats,” Endocrinology 1998 December; 139(12):5224-34; Jordan, V. C. et aZ., “Selective estrogen receptor modulation and reduction in risk of breast cancer, osteoporosis and coronary heart disease,” Natl Cancer Inst 2001 October; 93(19):1449-57; Guzzo J A., “Selective estrogen receptor modulators—a new age of estrogens in cardiovascular disease?,” Clin Cardiol 2000 January;23(1):15-7; Simoncini T, Genazzani A R., “Direct vascular effects of estrogens and selective estrogen receptor inodulators,” Curr Opin Obstet Gynecol 2000 June; 12(3):181-7.

Another embodiment of the invention is a method of treating or preventing the impairment of cognitive functioning or cerebral degenerative disorders in a mammal in need thereof by administering to the mammal a therapeutically effective amount of any of the compounds or pharmaceutical compositions described above. In models, estrogen has been shown to have beneficial effects on cognitive functioning, such as relieveing anxiety and depression and treating or preventing Alzheimer's disease. Estrogen affects the central nervous system by increasing cholinergic functioning, neurotrophin and neurotrophin receptor expression. Estrogen also increases glutamergic synaptic transmission, alters amyloid precursor protein processing and: provides neuroprotection. Thus, the estrogen receptor modulators of the present invention could be beneficial for improving cognitive functioning or treating mild cognitive impairment, attention deficit disorder, sleep disorders, irritability, impulsivity, anger management, multiple sclerosis and Parkinsons disease. See, Sawada, H and Shimohama, S, “Estrogens and Parkinson disease: novel approach for neuroprotection,” Endocrine. 2003 June;21(1):77-9; McCullough L D, and Hum, P D, “Estrogen and ischlemic neuroprotection: an integrated view,” Trends Endocrinol Metab. 2003 July;14(5):228-35; which arm hereby incorporated by reference in their entirety. The utility of SERMs to prevent the impairment of cognitive functioning is known in the art, see Yaffe, K., K. Krueger, S. Sarkar, et al. 2001. Cognitive function in postmenopausal women treated with raloxifene. N. Eng. J. Med. 344: 1207-1213.

Another embodiment of the invention is a method of treating or preventing depression in a mammal in need thereof by administering to the mammal a therapeutically effective amount of any of the compounds or pharmaceutical compositions described above. The utility of estrogens to prevent depression has been described in the art, see Carranza-Liram S., Valentino-Figueroa M L, “Estrogen therapy for depression in postmenopausal women.” Int J Gynnaecol Obstet 1999 April; 65(1):35-8. Specifically, estrogen receptor beta (ERβ) selective agonists would be useful in the treatment of anxiety or depressive illness, including depression, perimenopausal depression, post-partum depression, premenstrual syndrome, manic depression, anxiety, dementia, and obsessive compulsive behavior, as either a single agent or in combination with other agents. Clinical studies have demonstrated the efficacy of the natural estrogen, 17β-estradiol, for the treatment of various forms of depressive illness, see Schmidt P J, Nieman L, Danaceau M A, Tobin M B, Roca C A, Murphy J H, Rubinow D R. Estrogen replacement in perimenopause-related depression: a preliminary report. Am J Obstet Gynecol 183:414-20, 2000; and Soares C N, Almeida O P, Joffe H, Cohen L S. Efficacy of estradiol for the treatment of depressive disorders in perimenopausal women: a double-blind, randomized, placebo-controlled trial. Arch Gen Psychiatry. 58:537-8, 2001; which are hereby incorporated by reference. Bethea et al (Lu N Z, Shlaes T A, Gundlah C, Dziennis S E, Lyle R E, Bethea C L. Ovarian steroid action on tryptophan hydroxylase protein and serotonin compared to localization of ovarian steroid receptors in midbrain of guinea pigs. Endocrine 11:257-67, 1999, which is hereby incorporated by reference) have suggested that the anti-depressant activity of estrogen may be mediated via regulation of serotonin synthesis in the serotonin containing cells concentrated in the dorsal raphe nucleus.

Another embodiment of the invention is a method of treating or preventing anxiety in a mammal in need thereof by administering to the mammal a therapetitically effective amount of any of the compounds or pharmaceutical compositions described above. The contribution of estrogen receptors in the modulation of emotional processes, such as anxiety has been described in the art, see Krezel, W., et al., “Increased anxiety and synaptic plasticity in estrogen receptor beta-deficient mice.” Proc Natl Acad Sci USA 2001 October 9;98 (21):12278-82.

Another embodiment of the invention is a method of treating or preventing inflammation or inflammatory bowel disease. Inflammatory bowel diseases, including Crohn's Disease and ulceratie colitis, are chronic disorders in which the intestine (bowel) becomes inflamed, often causing recurring abdominal cramps and diarrhea. The use of estrogen receptor modulators to treat inflammation and inflammatory bowel disease has been described in the art, see Harris, H. A. et al., “Evaluation of an Estrogen Receptor-β Agonist in Animal Models of Human Disease,” Endocrinology, Vol. 144, No. 10 4241-4249.

Another embodiment of the invention is a method of treating or preventing hypertension. Estrogen receptor beta has been reported to have a role in the regulation of vascular function and blood pressure, see Zhu, et al., “Abnormal Vacular Function and Hypertension in Mice Deficient in Estrgoen Receptorβ ,” Science, Vol 295, Issue 5554, 505-508, 18 January 2002.

Another embodiment of the invention is a method of treating or preventing sexual dysfunction in males or females. The use of estrogen receptor modulators to treat sexual dysfunction has been described in the art, see Baulieu, E. et al., “Dehydroepiandrosterone (DHEA), DHEA sulfate, and aging: Contribution of the DHEAge Study to a scociobiomedical issue,” PNAS, Apr. 11, 2000, Vol. 97, No. 8, 4279-4282; Spark, Richard F., “Dehydroepiandrosterone: a springboard hormone for female sexuality,” Fertility and Sterility, Vol. 77, No. 4, Suppl 4, April 2002, S19-25.

Another embodiment of the invention is a method of treating or preventing retinal degeneration. Estrogen has been shown to have a beneficial effect of reducing the risk of advanced types of age-reated maculopathy, see Snow, K. K., et al., “Association between reproductive and hormonal factors and age-related maculopathy in postmenopausal women,” Americal Journal of Ophthalmology, Vol. 134, Issue 6, December 2002, pp. 842-48.

Exemplifying the invention is the use of any of the compounds described above in the preparation of a medicament for the treatment or prevention of osteoporosis in a mammal in need thereof. Still further exemplifying the invention is the use of any of the compounds described above in the preparation of a medicament for the treatment or prevention of: bone loss, bone resorption, bone fractures, metastatic bone disease or disorders related to estrogen functioning.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parentally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta actose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates arid crosslinked or amphipathic block copolymers of hydrogels.

The instant compounds are also useful in combination with known algents useful for treating or preventing bone loss, bone fractures, osteoporosis, metastatic bone disease, Paget's disease, periodontal disease, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate. Combinations of the presently disclosed compounds with other agents useful in treating or preventing the disorders disclosed herein are within the scope of the invention. A person of ordinary skill in the art would be able to discern which combinations of agents would be useful based on the particular characteristics of the drugs and the disease involved. Such agents include the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; selective serotonin reuptake inhibitors (SSRIs); an aromatase inhibitor; and the pharmaceutically acceptable salts and mixtures thereof. A preferred combination is a compound of the present invention and an organic bisphosphonate. Another preferred combination is a compound of the present invention and a cathepsin K inhibitor. Another preferred combination i s a compound of the present invention and an estrogen. Another preferred combination is a compound of the present invention and an androgen receptor modulator. Another preferred combination is a compound of the present invention and an osteoblast anabolic agent.

“Organic bisphosphonate” includes, but is not limited to, compounds of the chemical formula 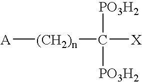
wherein n is an integer from 0 to 7 and wherein A and X are independently selected from the group consisting of H, OH, halogen, NH₂, SH, phenyl, C₁₋₃ alkyl, C₃₋₃₀ branched or cycloalkyl, bicyclic ring structure containing two or three N, C₁₋₃₀ substituted alkyl, C₁₋₁₀ alkyl substituted NH₂, C₃₋₁₀ branched or cycloalkyl substituted NH₂, C₁₋₁₀ dialkyl substituted NH₂, C₁₋₁₀ alkoxy, C₁₋₁₀ alkyl substituted thio, thiophenyl, halophenylthio, C₁₋₁₀ alkyl substituted phenyl, pyridyl, furanyl, pyrrolidinyl, imidazolyl, imidazopyridinyl, and benzyl, such that both A and X are not selected Prom H or OH when n is 0; or A and X are taken together with the carbon atom or atoms to which they are attached to form a C₁₋₁₀ ring.

In the foregoing chemical formula, the alkyl groups can be straight, branched, or cyclic, provided sufficient atoms are selected for the chemical formula. The C₁₋₃₀ substituted alkyl can include a wide variety of substituents, nonlimiting examples which include those selected from the group consisting of phenyl, pyridyl, furanyl, pyrrolidinyl, imidazonyl, NH₂, C₁₋₁₀ alkyl or dialkyl substituted NH₂, OH, SH, and C₁₋₁₀ alkoxy.

The foregoing chemical formula is also intended to encompass complex carbocyclic, aromatic and hetero atom structures for the A or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C₁₋₃₀ alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated.

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. For humans, an effective oral dose of bisphosphonate is typically from about 1.5 to about 6000 μg/kg body weight and preferably about 10 to about 2000 μg/kg of body weight. In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week. The bisphosphonates may also be administered monthly, ever six months, yearly or even less frequently, see WO 01/97788 (published Dec. 27, 2001) and WO 01/89494 (published Nov. 29, 2001).

“Estrogen” includes, but is not limited to naturally occurring estrogens [7-estradiol (E₂), estrone (E₁), and estriol (E₃)], synthetic conjugated estrogens, oral contraceptives and sulfated estrogens. See, Gruber C J, Tschugguel W, Schneeberger C, Huber J C., “Production and actions of estrogens” N Engl J Med 2002 January 31;346(5):340-52.

“Estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-nitrophenyl-hydrazone, and SH646.

“Cathepsin K inhibitors” refers to compounds which interfere with the activity of the cysteine protease cathepsin K. Nonlimiting examples of cathepsin K inhibitors can be found in PCT publications WO 00/55126 to Axys Pharmaceuticals and WO 01/49288 to Merck Frosst Canada & Co. and Axys Pharmaceuticals.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR® see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL® see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL® see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and 11.

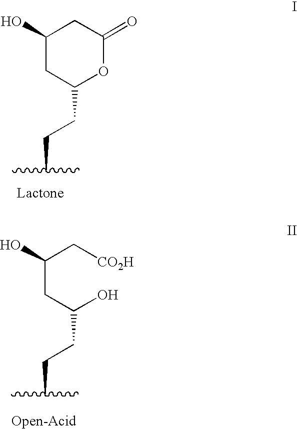

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically-acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, omithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not-limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅,αᵥβ₆, αᵥβ₈, α₁β₁, α₂β₁, α₅β₁, α₆β₁, and α₆β₄ integrins. H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact non-covalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

“An osteoblast anabolic agent” refers to agents that build bone, such as PTH. The intermittent administration of parathyroid hormone (PTH) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

Calcitonin is a 32 amino acid pepetide produced primarily by the thyroid which is known to participate in calcium and phosphorus metabolism. Calcitonin suppresses resorption of bone by inhibiting the activity of osteoclasts. Thus, calcitonin can allow osteoblasts to work more effectively and build bone.

“Vitamin D” includes, but is not limited to, vitamin D₃ (cholecalciferol) and vitamin D₂ (ergocalciferol), which are naturally occurring, biologically inactive precursors of the hydroxylated biologically active metabolites of vitamin D: 1α-hydroxy vitamin D; 25-hydroxy vitamin D, and la, 25-dihydroxy vitamin D. Vitamin D₂ and vitamin D₃ have the same biological efficacy in humans. When either vitamin D₂ or D₃ enters the circulation, it is hydroxylated by cytochrome P₄₅₀-vitamin D-25-hydroxylase to give 25-hydroxy vitamin D. The 25-hydroxy vitamin D metabolite is biologically inert and is further hydroxylated in the-kidney by cytochrome P450-monooxygenase, 25 (OH) D-1α-hydroxylase to give 1,25-dihydroxy vitamin D. When serum calcium decreases, there is an increase in the production of parathyroid hormone (PTH), which regulates calcium homeostasis and increases plasma calcium levels by increasing the conversion of 25-hydroxy vitamin D to 1,25-dihydroxy vitamin D.

1,25-dihydroxy vitamin D is thought to be reponsible for the effects of vitamin D on calcium and bone metabolism. The 1,25-dihydroxy metabolite is the active hormone required to maintain calcium absorption and skeletal integrity. Calcium homeostasis is maintained by 1,25 dihydroxy vitamin D by inducing monocytic stem cells to differentiate into osteoclasts and by maintaining calcium in the normal range, which results in bone mineralization by the deposition of calcium hydroxyapatite onto the bone surface, see Holick, M F, Vitamin D photobiology, metabolism, and clinical applications, In: DeGroot L, Besser H, Burger H G, eg al., eds. Endocrinology, 3^{rd} ed., 990-1013 (1995). However, elevated levels of 1α,25-dihydroxy vitamin D₃ can result in an increase of calcium concentration in the blood and in the abnormal control of calcium concentration by bone metabolism, resulting in hypercalcemia. 1α,25-dihydroxy vitamin D₃ also indirectly regulates osteoclastic activity in bone metabolism and elevated levels may be expected to increase excessive bone resorption in osteoporosis.

“Synthetic vitamin D analogues” includes non-naturally occurring compounds that act like vitamin D.

Selective Serotonin Reuptake Inhibitors act by increasing the amount of serotonin in the brain. SSRIs have been used successfully for a decade in the United States to treat depression. Non-limiting examples of SSRIs include fluoxetine, paroxetine, sertraline, citalopram, and fluvoxamine. SSRIs are also being used to treat disoreders realted to estrogen functioning, suchs as premenstrual syndrome and premenstrual dysmorphic disorder. See Sundstrom-Poromaa I, Bixo M, Bjorn I, Nordh O., “Compliance to antidepressant drug therapy for treatment of premenstrual syndrome,” J Psychosom Obstet Gynaecol 2000 December;21(4):205-11.

As used herein the term “aromatase inhibitor” includes compounds capable of inhibiting aromatase, for example commercially available inhibitors such as: aminoglutemide (CYTANDREN®), Anastrazole (ARIMIDEX®), Letrozole (FEMARA®), Formestane (LENATRON®), Exemestane (AROMASIN®), Atamestane (1-methylandrosta-1,4-diene-3,17-dione), Fadrozole (4-(5,6,7,8-Tetrahydroimidazo[1,5-a]pyridin-5-yl)-benzonitrile, monohydrochloride), Finrozole (4-(3-(4-Fluorophenyl)-2-hydroxy-1-(1H-1,2,4-triazol-1-yl)-propyl)-benzonitrile), Vorozole (6-[(4-chlorophenyl)-1H-1,2,4-triazol-1-ylmethyl]-1-methyl-1H-benzotriazole), YM-511 (4-[N-(4-bromobenzyl)-N-(4-cyanophenyl)amino]-4H-1,2,4-triazole) and the like.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a bisphosphonate, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

The present invention also encompasses a pharmaceutical composition useful in the treatment of osteoporosis or other bone disorders, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The compounds of the present invention can be used in combination with other agents useful for treating estrogen-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating cathepsin-mediated conditions includes in principle any combination with any pharmaceutical composition useful for treating disorders related to estrogen functioning.

The scope of the invention therefore encompasses the use of the instantly claimed compounds in combination with a second agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; a selective serotonin reuptake inhibitor; an aromatase inhibitor; and the pharmaceutically acceptable salts and mixtures thereof.

These and other aspects of the invention will be apparent from the teachings contained herein.

Definitions

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The term “bone resorption,” as used herein, refers to the process by which osteoclasts degrade bone.

The term “alkyl” shall mean a substituting univalent group derived by conceptual removal of one hydrogen atom from a straight or branched-chain acyclic saturated hydrocarbon (i.e., —CH₃, —CH₂CH₃, —CH₂CH₂CH₃, —CH(CH₃)₂, —CH₂CH₂CH₂CH₃, —CH₂CH(CH₃)₂, —C(CH₃)₃, etc.).

The term “alkenyl” shall mean a substituting univalent group derived by conceptual removal of one hydrogen atom from a straight or branched-chain acyclic unsaturated hydrocarbon (i.e., —CH═CH₂, —CH═CHCH₃, —C═C(CH₃)₂, —CH₂CH═CH₂, etc.).

The term “alkynyl” shall mean a substituting univalent group derived by conceptual removal of one hydrogen atom from a straight or branched-chain acyclic unsaturated hydrocarbon containing a carbon-carbon triple bond (i.e., —C≡CH, —C≡CCH₃, —C≡CCH(CH₃)₂, —CH₂C≡CH, etc.).

The term “acyl” shall mean a substituting univalent group derived by replacing two hydrogens on the attachment carbon of an “alkyl” group as described above with a carbonyl group (i.e., —COH, —COCH₃, —COCH₂CH₃, —COCH₂CH₂CH₃, —COCH(CH₃)₂, —COCH₂CH₂CH₂CH₃, —COCH₂CH(CH₃)₂, —COC(CH₃)₃, etc.).

The term “halo” shall include iodo, bromo, chloro and fluoro.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The present invention also includes N-oxide derivatives and protected derivatives of compounds of Formula L. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The compounds of the present invention may have asymmetric centers, chiral axes, and chiral planes (as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautojneric forms are intended to be encompassed by the scope of the invention, even though only one tautomeric structure is depicted. For example, any claim to compound A below is understood to include tautomeric structure B, and vice versa, as well as mixtures thereof.

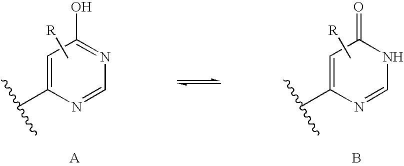

When any variable (e.g. R¹, R², R³ etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

In choosing compounds of the present invention, one of ordinary skill in the art will recognize that the various substituents, i.e. R¹, R², R³, R⁴ and R¹⁷ are to be chosen in conformity with well-known principles of chemical structure connectivity.

Representative compounds of the present invention typically display submicromolar affinity for alpha and/or beta estrogen receptors, and preferably agonize the beta estrogen receptor. Compounds of this invention are therefore useful in treating mammals suffering from disorders related to estrogen functioning.

The compounds of the present invention are available in racemic form or as individual enantiomers. For convenience, some structures are graphically represented as a single enantiomer but, unless otherwise indicated, is meant to include both racemic and enantiomerically pure forms. Where cis and trans sterochemistry is indicated for a compound of the present invention, it should be noted that the stereochemistry should be construed as relative, unless indicated otherwise. For example, a (+) or (−) designation should be construed to represent the indicated compound with the absolute stereochemistry as shown.

Racemic mixtures can be separated into their individual enantiomers by any of a number of conventional methods. These include, but are not limited to, chiral chromatography, derivatization with a chiral auxiliary followed by separation by chromatography or crystallization, and fractional crystallization of diastereomeric salts. Deracemization procedures may also be employed, such as enantiomeric protonation of a pro-chiral intermediate anion, and the like.

The compounds of the present invention can be used in combination with other agenis useful for treating estrogen-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

The compounds of the present invention can be prepared according to the following general schemes, using appropriate materials, and are further exemplified by the subsequent specific examples. The compounds illustrated in the examples are not, however, to be construed as forming the only genus that is considered as the invention. Those skilled in the art will readily understand that known variations of the conditions and processes of the following preparative procedures can be used to prepare these compounds. All temperatures are degrees Celsius unless otherwise noted.

Although the compounds of the present invention can be prepared by total synthesis, it is generally more practical to modify commercially available steroids. Dehydroepiandrosterone and androstenediol are especially convenient starting materials although other commercially available steroids may also be employed. Functionalization at C-19 can be accomplished by a number of methods known to those skilled in the art. One convenient method, which is illustrated in the following scheme, employs the 5,6-olefin of androstenediol as a handle to enable oxidation at C-19. The C-3 and C-17 hydroxyl groups of androstene diol are first protected as acetates, silyl ethers, THP ethers, or another suitable protecting group using standard procedures that are well known to those skilled in the art. Functionalization of the 5,6-olefin is accomplished by treating the protected diol intermediate with a bromine source such as N-bromoacetamide, N-Bromosuccinimide, and the like in the presence of an aqueous acid such as perchloric acid and the like. The product of this reaction has an axial hydroxyl group at C-6 of the steroid nucleus which serves as a handle for oxidation of the C-19 methyl group. One method by which this may be accomplished is by photolyzing a mixture of the alcohol, iodobenzene diacetate, and iodine in a hydrocarbon solvent such as cyclohexane. Reduction of the resulting cyclic ether with activated Zinc dust regenerates the 5,6-double bond and affords a 19-hydroxy steroid. The 19-hydroxy steroid can be oxidized to the key aldehyde intermediate A by a number of oxidation methods that are well known to those skilled in the art. One useful method for accomplishing this transformation involves reaction of the alcohol with tetrapropyl ammonium perruthenate (TPAP) and N-methyl morpholine N-oxide (NMO) in a solvent such as dichloromethane or chloroform and the like in the presence of molecular sieves. This aldehyde can serve as a substrate for many olefination reactions such as the Wittig, Peterson, or Tebbe reactions which are well known to those skilled in the art. Removal of the hydroxyl protecting groups using standard conditions then affords the final products as shown in the Scheme. 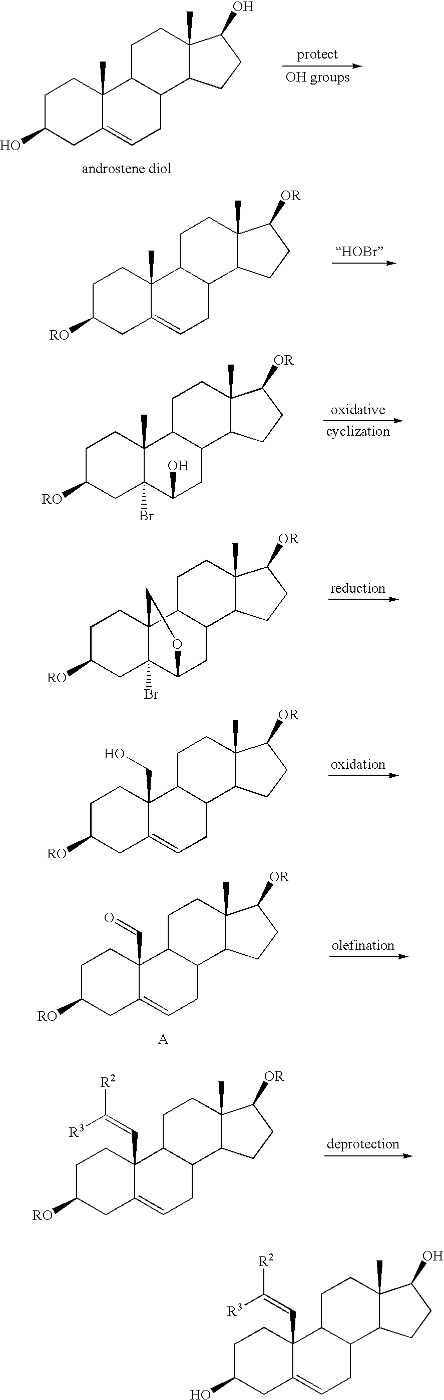
Carbon substituents at C-19 (R¹) may be introduced, as illustrated in the following Scheme, by reaction of the aldehyde intermediate A with a carbon nucleophile such as a Grignard or alkyl lithium reagent. Subsequent oxidation of the resulting secondary alcohol using one of the many available oxidation reagents which are well known to those skilled in the art affords the ketone intermediate B. Olefination of ketone B followed by removal of the hydroxyl protecting groups affords the C-19 substituted analogs. 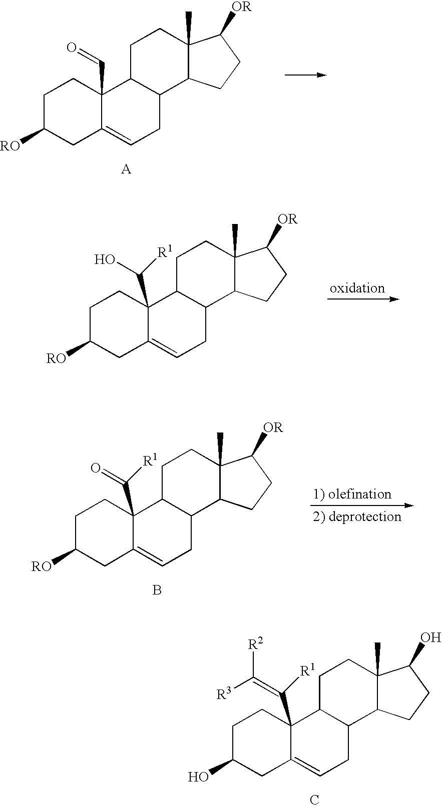
Carbon substituents at C-17 (R¹⁷) may be introduced, as illustrated in the following Scheme, by further reaction of the product (C) of the previous Scheme Selective protection of the less hindered hydroxyl group at C-3 of C with an appropriate protecting group such as a silyl ether, THP ether, and the like 5 followed by oxidation of the C-17 hydroxyl group using one of the many available oxidation reagents which are well known to those skilled in the art affords the ketone intermediate D. Reaction of the C-17 ketone with an appropriate carbon nucleophile such as a Grignard or alkyl lithium reagent introduces the R¹⁷ group. Subsequent removal of the C-3 hydroxyl protecting group using standard techniques affords the C-17 substituted analogs E.

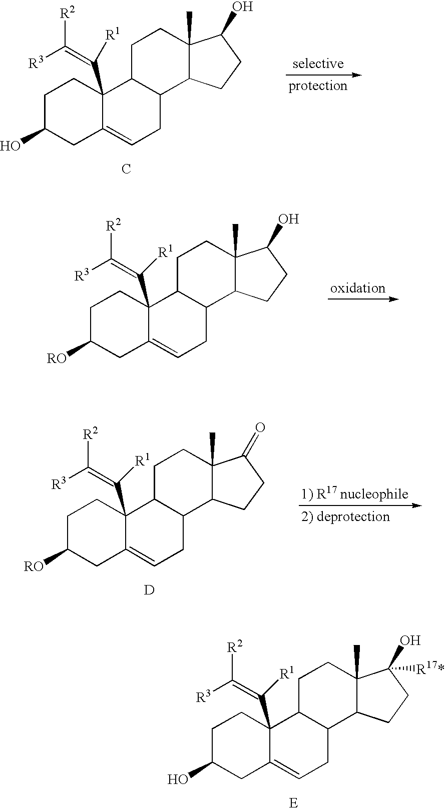

### EXAMPLES

### Preparation 1

### 3β,17β-ANDROST-5-ENE DIOL DIACETATE

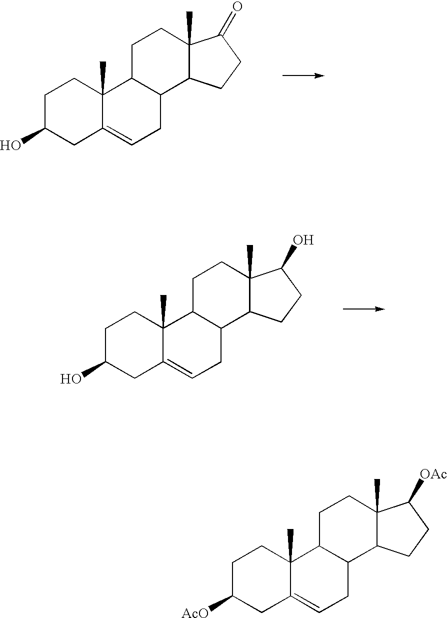 Step 1. 3β,17β-androst-5-ene diol:

Sodium borohydride (3.28 g, 0.0867 mol) was added in four equal portions (about 2 minutes apart) to a cold (0° C.) solution of dehydroepiandrosterone (25.0 g, 0.0867 mol) in methanol (870 mL). The cold bath was removed and the cloudy white mixture was stirred at room temperature for 90 minutes. The reaction mixture was cooled in an ice bath as 2N HCl (173 mL, 0.346 mol) was added dropwise. The resulting mixture was concentrated under vacuum to a wet white solid. Water (500 mL) was added and the mixture was sonicated and filtered. The collected solid was washed with water (100 mL) and dried in a vacuum dessicator overnight to afford the title compound as a white solid.

Step 2. 3β,17β-androst-5-ene diol diacetate:

Acetic anhydride (19.5 mL, 0.2 mol) was added to a solution of 3β,17β-androst-5-ene diol (15.0 g, 0.05165 mol) in pyridine (200 mL) (note: the addition was mildly exothemic) then 4-dimethylamino-pyridine (0.63 g, 0.00516 mol) was added. The resulting yellow solution was stirred at room temperature for 5.5 hours then most of the solvent was removed under vacuum. The residual yellow-white sludge was partitioned between ethyl acetate (450 mL) and 1N HCl (450 mL). The organic layer was washed with 5% aqueous sodium bicarbonate (200 mL) then dried over magnesium sulfate, filtered, and evaporated to an off-white solid. This crude product was recrystallized from hexane (500 mL) to afford the title compound as a white crystalline solid. Concentration of the mother liquor from the recrystallization afforded an off-white solid which could be recrystallized to afford a second crop of product.

### Preparation 2

### 19-OXO-30β,17β-ANDROST-5-ENE DIOL DIACETATE

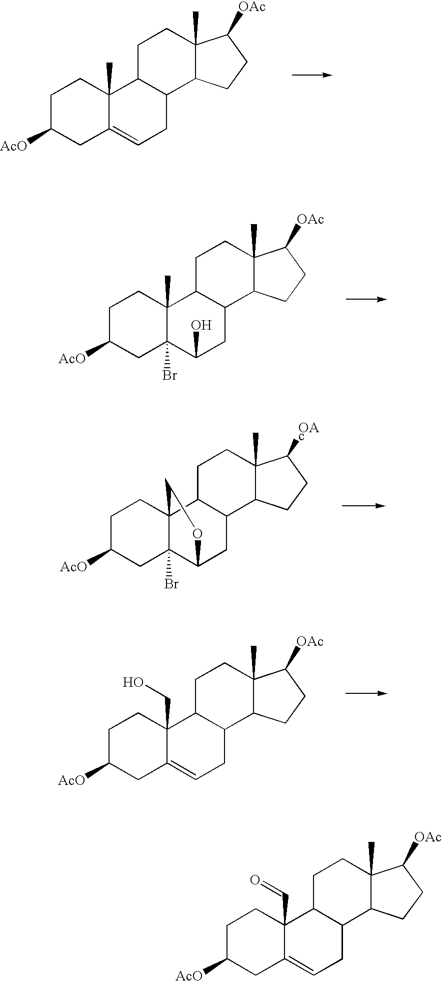 Step, 1. 5β-bromo-6β-hydroxy-3β,17β-androstane diol diacetate:

A solution of 70% perchloric acid (0.79 mL) in water (6.8 mL) was added to a solution of 3β,17β-androst-5-ene diol diacetate (4.17 g, 0.011 mol) in dioxane (56 mL) and water (3.4 mL) at 5 ° C. N-bromoacetamide (2.25 g, 0.016 mol) was added in small portions over a 20 minute period. The resulting mixture was stirred at 5° C. for 30 minutes then stirred at room temperature for 30 minutes then poured into water containing 0.5 mL of 1% sodium thiosulfate solution. The suspension was adjusted to pH 8 by addition of saturated aqueous sodium bicarbonate solution then extracted with ethyl acetate. The organic layer was washed with brine, dried over magnesium sulfate, filtered, and concentrated under vacuum to afford a white foam. The residue was combined with 0.296 g of crude product from an earlier batch and purified by recrystallization from acetone/hexane to afford the title compound as a white solid containing about 20% of the isomeric 5β,6β by-product.

Step 2. 5β-bromo-6β,19-epoxy-3β,17β-androstane diol diacetate:

Iodobenzene diacetate (1.23 g, 0.0057 mol) was added to a suspension of the product of step 1 (1.8 g, 0.0038 mol) in cyclohexane (250 mL) then iodine (0.97 g, 0.0038 mol) was added. The resulting mixture was irradiated with a 200 W sun lamp for 45 minutes (note: the temperature of the mixture rose to about 80° C. during this time). The reaction mixture was cooled to room temperature and poured into ice/water. The resulting mixture was extracted with ether. The organic layer was washed with 2% aqueous sodium thiosulfate and water then dried over magnesium sulfate, filtered, and concentrated under vacuum. The residue was recrystallized from hexane to afford an off-white solid.

Step 3. 3β,17β,19-androst-5-ene triol 3,17-diacetate:

A mixture of activated zinc dust (11.1 g, 0.17 mol; activated before use by brief treatment with aqueous HCl followed by sequential washing with water and and acetone and drying under vacuum) and the product of step 2 (1.50 g, 0.0032 mol) in tetrahydrofuran (75 mL) and water (7.5 mL) was stirred at 65° C. for 1 hour. The reaction mixture was cooled to room temperature and filtered. The collected solid was washed with ether then the combined filtrate was washed with water, dried over magnesium sulfate, filtered, and concentrated under vacuum to afford a pale yellow foam. The residue was recrystallized from acetone/hexane to afford the title compound as a pale yellow solid. Concentration and recrystallization of the mother liquor afforded a second crop of less pure product as a pale yellow solid.

Step 4. 19-oxo-3β,17β-androst-5-ene diol diacetate:

Activated 4A molecular sieves (4.2 g) were added to a cold (0° C.) solution of the product of step 3 (0.500 g, 0.00128 mol) and N-methylmorpholine N-oxide (NMO, 2.43 g, 0.0207 mol) in dichloromethane (10 mL). The resulting mixture was stirred at 0° C. for 15 minutes then tetrapropylamonium perruthenate (0.030 g, 0.0000854 mol) was added. The resulting mixture was stirred at 0° C. for 90 minutes then diluted with ether and filtered The collected solid was washed with ether. The combined filtrate was washed sequentially with aqueous sodium sulfite, and aqueous copper sulfate, then dried over magnesium sulfate, filtered, and concentrated under vacuum to afford a white solid. The residue was purified by flash chromatography on silica gel eluted with 95:5 dichloromethane:ethyl acetate to afford the title compound as a pale yellow solid.

### Preparation 3

### 19-OXO-3β,17β-ANDROST-5-ENE DIOL 3,17-BIS-O-TETRAHYDROPYRANYL ETHER

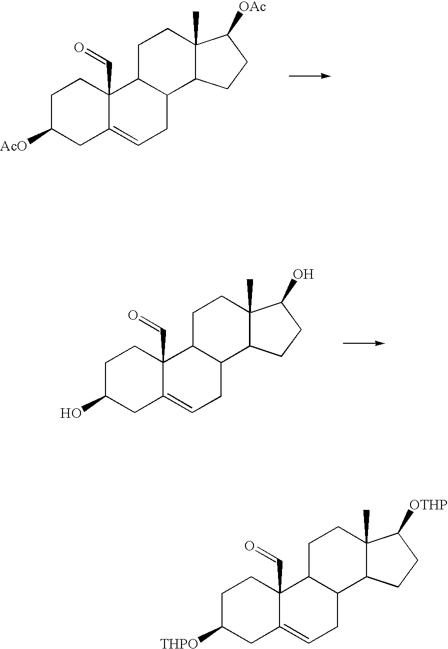 Step 1: 19-oxo-3β,17β-androst-5-ene diol:

A mixture of 19-oxo-3β,17β-androst-5-ene diol diacetate (0.40 g, 0.00103 mol) and 10% potassium hydroxide in methanol (20 mL) was stirred at rom temperature for 6 hours. Most of the solvent was removed under vacuum and the residue was partitioned between water and 5% methanol in dichloromethane. The aqueous layer was extracted with dichloromethane (2×) and the combined organic layers were dried over magnesium sulfate, filtered and concentrated under vacuum to afford the title compound as an off-white solid.

Step 2: 19-oxo-3β,17β-androst-5-ene diol 3,17-bis-O-tetrahydropyranyl ether:

A mixture of 19-oxo-3β,17β-androst-5-ene diol (0.292 g, 0.00096 mol), dihydropyran (1.0 mL, 0.011 mol), and pyridinium tosylate (0.061 g, 0.00024 mol) in tetrahydrofuran (12 mL) was stirred at room temperature overnight. Most of the solvent was removed under vacuum and the residue was partitioned between water and dichloromethane. The organic layer was dried over magnesium sulfate, filtered and concentrated under vacuum. The residue was recrystallized from methanol/water to afford the title compound as a yellow solid. NMR analysis indicated a mixture of diastereomers.

### Example 1

### 19-NOR-10β-VINYL-3β,17β-ANDROST-5-ENE DIOL

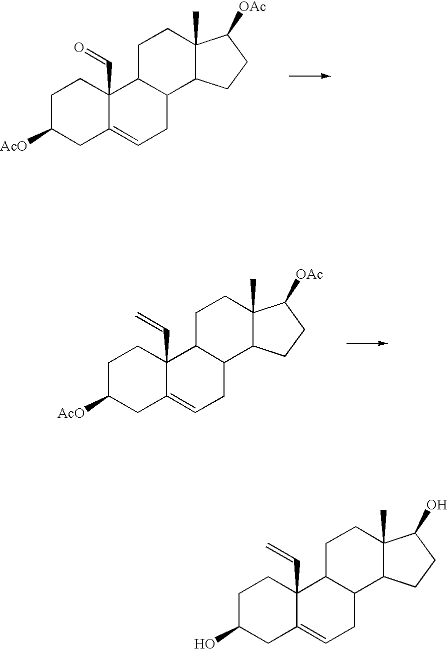 Step 1. 19-nor-10β-vinyl-3β,17β-androst-5-ene diol diacetate:

nButyllithium (0.80 mL of 1.63 M hexane solution, 0.0013 mmol) was added to a cold (0° C.) suspension of methyl triphenylphosphonium bromide (0.503 g, 0.0014 mol) in tetrahydrofuran (5 mL). The resulting mixture was stirred at 0° C. for 1 hour then a solution of 19-oxo-3β,17β-androst-5-ene diol diacetate (0.182 g, 0.000469 mol) in tetrahydrofuran (2 mL) was added. The mixture was stirred at 0° C. for 4 hours then the reaction was quenched by addition (f saturated aqueous ammonium chloride. The resulting mixture was extracted with ethyl acetate (2×) and the combined extracts were dried over magnesium sulfate, filtered, and concentrated under vacuum to afford a gummy tan solid. NMR analysis indicated a mixture of the product and deacetylated product. In order to facilitate purification, the crude product mixture was reacetylated (dissolved in dichloromethane (2 mL) then added 4-dimethylamino pyridine (a few crystals), pyridine (0.020 mL), and acetic anhydride (0.074 mL, 0.00074 mol); stirred at room temperature overnight then diluted with ethyl acetate, washed sequentially with dilute aqueous HCl, water, and brine then dried over magnesium sulfate, filtered, and concentrated under vacuum to afford a gummy amber solid. The crude product was purified by flash chromatography on silica gel eluted with 9:1 hexane:ethyl acetate to afford the title compound as a colorless oil.

Step 2. 19-nor-10β-vinyl-3β,17β-androst-5-ene diol:

A mixture of the product of step 1 (0.193 g, 0.0005 mol, combined product of several batches) and 1N sodium hydroxide (2 mL, 0.002 mol) in methanol (5 mL) was stirred at room temperature for 3 hours then neutralized by addition of 1N HCl. Most of the solvent was removed under vacuum and the residue was diluted with water and filtered. The collected solid was washed with water then dissolved in methanol and filtered to remove insoluble material. The methanol was removed under vacuum and the residue was recrystallized from acetone/hexane to afford the title compound as a white solid. Concentration and recrystallization of the mother liquor afforded a second crop of the title compound as a white solid. Selected ¹H NMR data: (CDCl₃, 600 MHz) δ 5.65 (1H, dd, J=11, 17 Hz), 5.28 (1H, dd, J=2, 11 Hz), 4.95 (1H, dd, J=2, 17 Hz), 3.61 (1H, t, J=9 Hz), 0.65 (3H, s)

### Example 2

### 19-NOR-10β-(CIS-2-METHYL-VINYL)-3β,17β-ANDROST-5-ENE DIOL

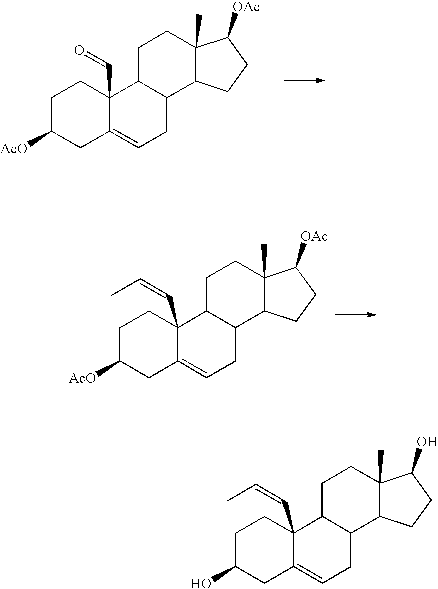 Step 1. 19-nor-10β-(cis-2-methyl-vinyl)-3β,17β-androst-5-ene diol diacetate:

nButyllithium (0.60 mL of 1.63 M hexane solution, 0.00096 mmol) was added to a cold (0° C.) suspension of ethyl triphenylphosphonium bromide (0.373 g, 0.001 mol) in tetrahydrofuran (6 mL). The resulting mixture was stirred at 0° C. for 1 hour then a solution of 19-oxo-3β,17β-androst-5-ene diol diacetate (0.130 g, 0.000335 mol) in tetrahydrofuran (2 mL) was added. The mixture was stirred at 0° C. for 4 hours then the reaction was quenched by addition c)f saturated aqueous ammonium chloride. The resulting mixture was extracted with ethyl acetate (2×) and the combined extracts were dried over magnesium sulfate, filtered, and concentrated under vacuum to afford a gummy tan solid. NMR analysis indicated a mixture of the product and deacetylated product. In order to facilitate purification, the crude product mixture was reacetylated (dissolved in dichloromethane (2 mL) then added 4-dimethylamino pyridine (a few crystals), pyridine (0.050 mL), and acetic anhydride (0.053 mL, 0.00053 mol); stirred at room temperature overnight then diluted with ethyl acetate, washed sequentially with dilute aqueous HCl, water, and brine then dried over magnesium sulfate, filtered, and concentrated under vacuum to afford a gummy amber solid. The crude product was purified by flash chromatography on silica gel eluted with 9:1 hexane:ethyl acetate to afford the title compound as an off-white solid. A small amount of impure trans isomer was isolated as a by-product.

Step 2. 19-nor-10β-(cis-2-methyl-vinyl)-3β,17β-androst-5-ene diol:

A mixture of the product of step 1 (0.025 g, 0.0000625 mol) and 1N sodium hydroxide (0.25 mL, 0.00025 mol) in methanol (1 mL) was stirred at room temperature for 3 hours then neutralized by addition of 1N HCl. Most of the solvent was removed under vacuum and the residue was diluted with water and filtered. The collected solid was washed with water then dissolved in methanol and filtered to remove insoluble material. The methanol was removed under vacuum and the residue was recrystallized from acetone/hexane to afford the title compound as a white solid. Selected ¹H NMR data: (CDCl₃, 600 MHz) δ 5.61 (1H, dq, J=12, 7 Hz), 5.08 (1H, dq, J=12, 2 Hz), 3.63 (1H, t, J=9 Hz), 1.70 (3H, dd, J=2, 7 Hz), 0.70 (3H, s)

### Example 3

### 19-NOR-10β-(CIS-2-CHLORO-VNYL)-3β, 17β-ANDROST-5-ENE DIOL DIOL AND 19-NOR-10β-(TRANS-2-CHLORO-VINYL)-3β,17β-ANDROST-5-ENE DIOL

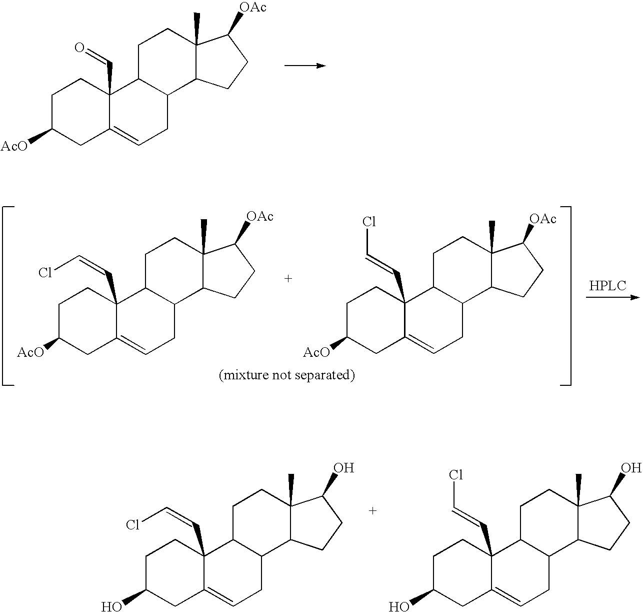 Step 1. 19-nor-10β-(cis-2-chloro-vinyl)-3β,17β-androst-5-ene diol diacetate and 19-nor-10β-(trans-2-chloro-viny)-3β,17β-androst-5-ene diol diacetate:

nButyllithium (0.30 mL of 1.63 M hexane solution, 0.000489 mmol) was added to a cold (−45° C.) suspension of chloromethyl triphenylphosphonium bromide (0.170 g, 0.00049 mol) in tetrahydrofuran (7 mL). The resulting mixture was stirred at −45° C. for 1 hour then a solution of 19-oxo-3β,17β-androst-5-ene diol diacetate (0.050 g, 0.000129 mol) in tetrahydrofuran (2 mL) was added. The mixture was stirred at −45° C. for 10 minutes then allowed towarm to room temperature. The resulting mixture was stirred at room temperature for 3 hours then the reaction was quenched by addition of saturated aqueous ammonium chloride. The resulting mixture was extracted with ethyl acetate (2×) and the combined extracts were dried over magnesium sulfate, filtered, and concentrated under vacuum to afford a gummy tan solid. The crude product was purified by flash chromatography on silica gel eluted initially with 9:1 hexane:ethyl acetate with the eluent gradually changed to 3:2 hexane:ethyl acetate (gradient elution) to afford the title compound as a colorless oil as a 4:1 cis:trans mixture of olefin isomers.

Step 2. 19-nor-10β-(cis-2-chloro-vinyl)-31β,17β-androst-5-ene diol and 19-nor-10β-(trans-2-chloro-vinyl)-3β,17β-androst-5-ene diol:

A mixture of the product of step 1 (0.038 g, 0.00009 mol) and 1N sodium hydroxide (0.50 mL, 0.0005 mol) in methanol (1 mL) was stirred at room temperature overnight then neutralized by addition of 1N HCl. Most of the solvent was removed under vacuum and the residue was suspended in water and extracted with dichloromethane (3×). The combined extracts were dried over magnesium sulfate, filtered, and concentrated under vacuum to afford a colorless oil. The crude product was combined with the product of three other batches and purified by flash chromatography on silica gel eluted with 4:1 dichloromethane:ethyl acetate to afford the title compound as a mixture of olefin isomers. The olefin isomers were further purified by preparative HPLC on a Chiralcel OJ column eluted with 91:9 heptane:ethanol to afford the title compounds as single olefin isomers (olefin geometry determined by NMR analysis). Selected ¹H NMR data for cis isomer: (CDCl₃, 600 MHz) δ 6.28 (1H, d, J=8 Hz), 5.56 (1H, d, J=8 Hz), 3.55 (1H, t, J=9 Hz), 0.70 (3H, s); selected ¹H NMR data for trans isomer: (CDCl₃, 600 MHz) δ 5.87 (1H, d, J=14 Hz), 5.84 (1H, d, J=14 Hz), 3.55 (1H, t, J=9 Hz), 0.66 (3H, s)

### Example 4

### 19-NOR-10β-CIS-2-BROMO-VINYL)-3β,17β-ANDROST-5-ENE DIOL

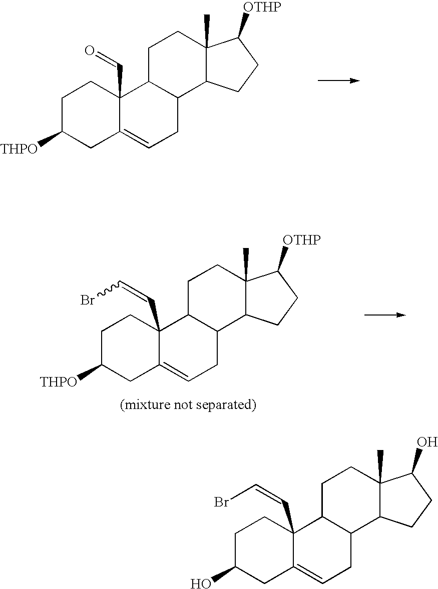 Step 1. 19-nor-10β-(cis-2-bromo-vinyl)-3β, 17β-androst-5-ene diol-3,17-bis-O-tetrahydropyranyl ether and 19-nor-10β-(trans-2-bromo-vinyl)-3β,17β-androst-5-ene diol-3,17-bis-O-tetrahydropyranyl ether:

Potassium tert-butoxide (14.0 mL of 1M THF solution, 14 mmol) was added to a cold (0° C.) suspension of bromomethyl triphenylphosphonium bromide (6.45 g, 14.8 mmol) in tetrahydrofuran (6 mL). The resulting mixture was stirred at 0° C. for 2 hours then 19-oxo-3β,17β-androst-5-ene diol 3,17-bis-O-tetrahydropyranyl ether (1.0 g, 2.1 mmol) was added. The mixture was stirred at reflux overnight then cooled to room temperature and quenched by addition of saturated aqueous ammonium chloride. The resulting mixture was extracted with ethyl acetate (3×) and the combined extracts were dried over magnesium sulfate, filtered, and concentrated under vacuum to afford a dark amber gum. The crude product was purified by flash chromatography on silica gel eluted initially with 9:1 hexane:ethyl acetate with the eluent gradually changed to 4:1 hexane:ethyl acetate (gradient elution) to afford the title compound as a colorless oil which was a mixture of cis and trans olefin isomers. Additional material was isolated as a mono-THP ether and could be recycled to the bis-THP ether if desired.

Step 2. 19-nor-10β-(cis-2-bromo-vinyl)-3β,17β-androst-5-ene diol:

A solution of the product of step 1 (300 mg, 0.55 mmol; combined product of several runs) and pyridinium tosylate (360 mg, 1.4 mol) in methanol (5 mL) was stirred at room temperature overnight then diluted with ethyl acetate, washed with water and saturated aqueous sodium chloride. The combined extracts were dried over magnesium sulfate, filtered, and concentrated under vacuum to afford a tan solid. The crude product was purified by flash chromatography on silica gel eluted with 3:2 hexane:ethyl acetate to afford the title compound (olefin geometry determined by NMR analysis). Selected ¹H NMR data: (CDCl₃, 600 MHz) δ 6.34 (1H, d, J=8 Hz), 5.90 (1H, d, J=8 Hz), 3.63 (1H, t, J=9 Hz), 0.72 (3H, s). The less active trans product could also be isolated from the crude product although derivatization and extensive chromatograph was required.

### Example 5

### 19-NOR-10β-VINYL-17α-METHYL-3β,17β-ANDROST-5-ENE DIOL

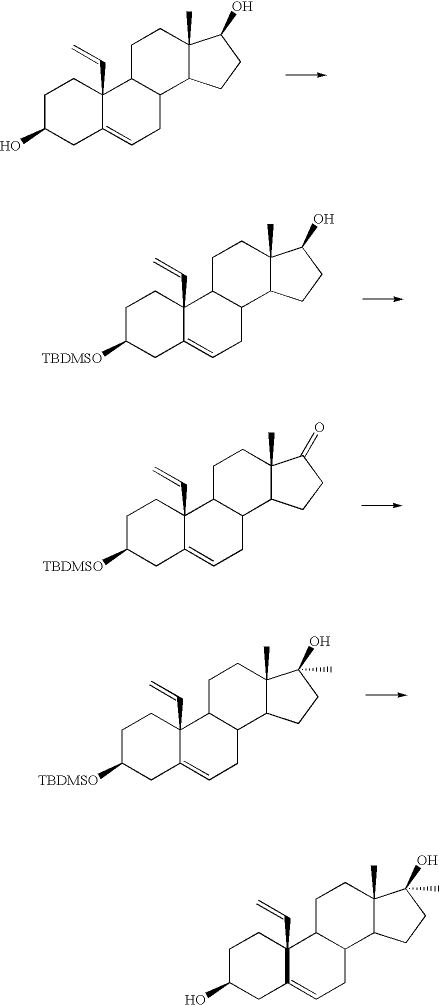 Step 1. 3-βtert-butyldimethylsilyloxy-19-nor-10β-viny-17βhydroxy-androst-5-ene:

A solution of 19-nor-10β-vinyl-3β,17β-androst-5-ene diol (119 mg, 0.39 mmol), imidazole (170 mg, 2.5 mmol) and tert-butyldimethylsilyl chloride (219 mg, 1.4 mol) in dimethylformamide (3 mL) was stirred at room temperature for 30 minutes then partitioned between ethyl acetate and water. The aqueous layer was extracted with ethyl acetate and the combined extracts were dried over magnesium sulfate, filtered, and concentrated under vacuum The crude product was purified by flash chromatography on silica gel eluted with 3:1 hexane:ethyl acetate to afford the title compound as a white solid.

Step 2. 3-β-tert-butyldimethylsilyloxy-19-nor-10β-vinyl-17-oxo-androst-5-ene:

A solution of the product of step 1 (74 mg, 0. 18 mmol) and N-methylmorpholine-N-oxide (330 mg, 2.8 mmol) in dichloromethane (3 mL) was stirred with activated 4A molecular sieves at room temperature as tetra-propylammonium perruthenate (5.9 mg, 0.017 mmol) was added. The resulting mixture was stirred at room temperature overnight then diluted with ether and filtered. The filtrate was concentrated under vacuum and the crude product was purified by flash chromatography on silica gel eluted with 9:1 hexane:ethyl acetate to afford the title compound as a white solid.

Step 3. 3-O-tert-butyldimethylsilyloxy-19-nor-10β-vinyl-17-α-methyl-17-β-hydroxy-androst-5-ene:

A solution of the product of step 2 (26 mg, 0.062 mmol) in tetrahydrofuran (0.6 mL) was added to a solution of methylmagnesium iodide (0.2 mL of 3 M solution in tetrahydrofuran, 0.6 mmol. The resulting mixture was stirred at room temperature overnight then quenched with water and partitioned between ethyl acetate and saturated aqueous sodium chloride. The aqueous layer was extracted with ethyl acetate (2×) and the combined organic layers dried over magnesium sulfate, filtered, and concentrated under vacuum. The crude product was purified by flash chromatography on silica gel eluted with 3:1 hexane:ethyl acetate to afford the title compound as a white solid.

Step 4. 19-nor-10β-vinyl-17-α-methyl-3β,17-β-androst-5-ene diol:

A solution of the product of step 3 (14 mg, 0.032 mmol) and tetrabutylammonium fluoride (0.2 mL of 1 M solution in tetrahydrofuran, 0.2 mmol) in tetrahydrofuran (0.4 mL) was stirred at room temperature overnight. The solvent was removed under vacuum and the crude product was purified by flash chromatography on silica gel eluted with 3:1 hexane:ethyl acetate to afford the title compound. Selected ¹H NMR data: (CDCl₃, 600 MHz) δ 5.67 (1H, dd, J 11, 18 Hz), 5.29 (1H, dd, J=2, 11 Hz), 4.96 (1H, dd, J=2, 18 Hz), 1.20 (3H, s), 0.77 (3H, s).

### Example 6

### 19-NOR-10β-VINYL-17α-ETHYNYL-3β,17β-ANDROST-5-ENE DIOL

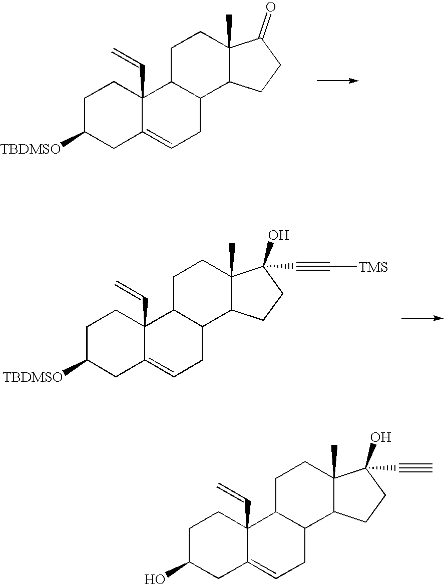 Step 1. 3-tert-butyldimethylsilyloxy-19-nor-10β-vinyl-17-α-(2-trimethylsilylethynyl)-17-β-hydroxy-androst-5-ene:

tert-Butyllithium (0.37 mL of 1.7 M pentane solution, 0.63 mmol) was added to a solution of trimethylsilylacetylene (0.1 mL, 0.71 mmol) in dry tetrahydrofuarn (0.2 mL). A solution of 3-β-tert-butyldimethylsilyloxy-19-nor-10β-vinyl-17-oxo-androst-5-ene (see step 2 of example 5) (26 mg, 0.062 mmol) in tetrahydrofuran (0.6 mL) was then added. The resulting mixture was stirred at room temperature overnight then quenched with water and extracted with 5% methanol in dichloromethane (3×). The combined organic layers dried over magnesium sulfate, filtered, and concentrated under vacuum. The crude product was identified by NMR and used without purification in the next step.

Step 2. 19-nor-10β-vinyl-17-α-ethynyl-3-13β,17β-13-androst-5-ene diol:

A solution of the product of step 1 (32 mg, 0.062 mmol) and tetrabutylammonium fluoride (0.3 mL of 1 M solution in tetrahydrofuran, 0.3 mmol) in tetrahydrofuran (2 mL) was stirred at room temperature for two hours then additional tetrabutylammonium fluoride (0.6 mL of 1 M solution in tetrahydrofuran, 0.6 mmol) was added and the solution was stirred at room temperature overnight. The solvent was removed under vacuum and the crude product was purified by flash chromatography on silica gel eluted with 3:1 hexane:ethyl acetate to afford the title compound. Selected ¹H NMR data: (CDCl₃, 600 MHz) δ 5.66 (1H, dd, J=11, 18 Hz), 5.28 (1H, dd, J=2, 11 Hz), 4.96 (1H, dd, J=2, 18 Hz), 2.56 (1H, s), 0.76 (3H, s).

### Example 7

### 3β-HYDROXY-17β-METHOXY-19-NOR-10β-VINYL-ANDROST-5-ENE

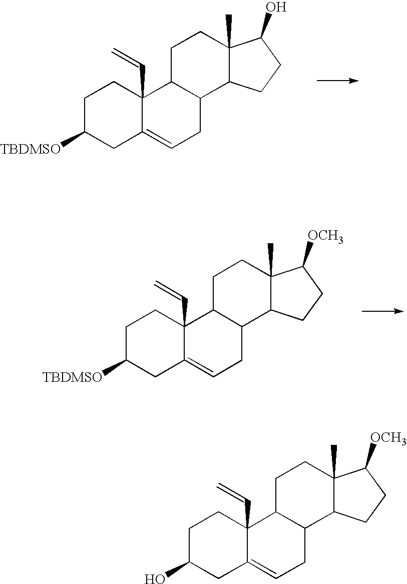 Step 1. 3-β-tert-butyldimethylsilyloxy-17β-methoxy-19-nor-10β-vinyl-androst-5-ene:

Sodium hydride (14 mg of 60% oil dispersioin, 0.36 mmol) is added to a solution of 3-β-tertbutyldimethylsilyoxy-17β-hydroxy-19-nor-10β-vinyl-androst-5-ene (75 mg, 0.18 mmol) in dry dimethylformamide (2 mL). The resulting mixture is stirred at room temperature for 10 minutes then iodomethane (255 mg, 1.8 mmol) is added. The resulting mixture is stirred at 55° C. overnight then partitioned between ethyl acetate and water. The aqueous layer is extracted with ethyl acetate and the combined extracts are dried over magnesium sulfate, filtered, and concentrated under vacuum The crude product is used without purification in the next step.

Step 2. 3β-hydroxy-17β-methoxy-19-nor-10β-vinyl-androst-5-ene:

A solution of 3-O-tert-butyldimethylsilyloxy-17β-methoxy-19-nor-10β-vinyl-androst-5-ene (43 mg, 0.1 mmol) and tetrabutylammonium fluoride (0.5 mL of 1 M solution in tetrahydrofuran, 0.5 mmol) in tetrahydrofuran (1.5 mL) is stirred at room temperature overnight. The solvent is removed under vacuum and the crude product is purified by flash chromatography on silica gel eluted with 3:1 hexane:ethyl acetate to afford the title compound.

Estrogen Receptor Binding Assay

The estrogen receptor ligand binding assays are designed as scintillation proximity assays employing the use of tritiated estradiol and recombinant expressed estrogen receptors. The full length recombinant human ER-α and ER-β proteins are produced in a bacculoviral expression system. ER-α or ER-β extracts are diluted 1:400 in phosphate buffered saline containing 6 mM α-monothiolglycerol. 200 μL aliquots of the diluted receptor preparation are added to each well of a 96-well Flashplate. Plates are covered with Saran Wrap and incubated at 4° C. overnight.

The following morning, a 20 ul aliquot of phosphate buffered saline containing 10% bovine serum albumin is added to each well of the 96 well plate and allowed to incubate at 40° C. for 2 hours. Then the plates are washed with 200 ul of buffer containing 20 mM Tris (pH 7.2), 1 mM EDTA, 10% Glycerol, 50 mM KCl, and 6 mM α-monothiolglycerol. To set up the assay in these receptor coated plates, add 178 ul of the same buffer to each well of the 96 well plate. Then add 20 ul of a 10 nM solution of ³H-estradiol to each well of the plate.

Test compounds are evaluated over a range of concentrations from 0.01 nM to 1000 nM. The test compound stock solutions should be made in 10% DMSO at 100× the final concentration desired for testing in the assay. The amount of DMSO in the test wells of the 96 well plate should not exceed 1%. The final addition to the assay plate is a 2 ul aliquot of the test compound which has been made up in 100% DMSO. Seal the plates and allow them to equilibrate at room temperature for 3 hours. Count the plates in a scintillation counter equipped for counting 96 well plates.

The compounds of Examples 1-3 exhibit binding affinities to the estrogen receptor α-subtype in the range of IC₅₀=75 to >10000 nm, and to the estrogen receptor β-subtype in the range of IC₅₀=5 to 250 nm.

Pharmaceutical Composition

As a specific embodiment of this invention, 25 mg of compound of Example 1, is formulated with sufficient finely divided lactose to provide a total amount of 580 to 590 mg to fill a size 0, hard-gelatin capsule.

What is claimed is:

1. A method of treating a disease in a mammal in need thereof by administering to the mammal a therapeutically effective amount of a compound of the formula: 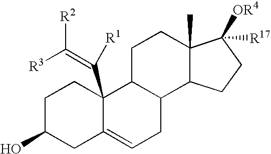
wherein R¹ is hydrogen, halo, C₍₁₋₃₎ alkyl, CO₂C₍₁₋₃₎alkyl, or cyano;
R² is hydrogen, halo, C₍₁₋₃₎ alkyl, CO₂C₍₁₋₃₎alkyl, or cyano;
R³ is hydrogen, halo, C₍₁₋₃₎ alkyl, CO₂C₍₁₋₃₎alkyl, or cyano;
R⁴ is hydrogen or C₍₁₋₃₎ alkyl;
R¹⁷ is hydrogen, C₍₁₋₅₎ alkyl, C₍₁₋₅₎ acyl, C₍₂₋₅₎ alkenyl, or C₍₂₋₅₎ alkynyl;
or a pharmaceutically acceptable salt or stereoisomer thereof;
wherein said disease is: bone loss, bone fractures, osteoporosis, metastaic bone disease, Paget's disease, periodontal disease, cartilage degeneration, endometriosis, uterine fibroid disease, hot flashes, cardiovascular disease, impairment of cognitive functioning, cerebral degenerative disorders, restenosis, gynecomastia, vascular smooth muscle cell proliferation, obesity, incontinence, anxiety, depression, perimenopausal depression, post-partum depression, premenstrual syndrome, manic depression, anxiety, dementia, obsessive compulsive behavior, attention deficit disorder, sleep disorders, irritability, impulsivity, anger management, multiple sclerosis and Parkinson's disease, inflammation, inflammatory bowel disease, sexual dysfunction, hypertension, retinal degeneration or an estrogen dependent cancer.

2. The method of claim 1 wherein
R¹ is hydrogen, halo, methyl or cyano;
R² is hydrogen, halo, methyl or cyano;
R³ is hydrogen, halo, methyl or cyano;
R⁴ is hydrogen or methyl;
R¹⁷ is hydrogen, C₍₁₋₃₎ alkyl, C₍₂₋₃₎ acyl, C₍₂₋₃₎ alkenyl, or C₍₂₋₃₎ alkynyl.

3. The method of claim 1 wherein the disease is hot flashes.

4. The method of claim 1 wherein the disease is depression.

5. The method of claim 1 wherein the disease is an estrogen dependent cancer.

6. The method of claim 1 wherein the compound is selected from
19-nor-10β-vinyl-3β,17β-androst-5-ene diol;
19-nor-10β-vinyl-30-hydroxy-17β-methoxy-androst-5-ene
17α-ethynyl-19-nor-10β-vinyl-3β,17β-androst-5-ene diol;
17α-vinyl-19-nor-10β-vinyl-3β,17₍₁₋₃₎ -androst-5-ene diol;
17α-methyl-19-nor-10β-vinyl-3β,17β-androst-5-ene diol;
19-nor-10β-(1-methyl-vinyl)-3β,17β-androst-5-ene diol;
19-nor-10β-(cis-2-methyl-vinyl)-3β,17β-androst-5-ene diol;
19-nor-10β-(trans-2-methyl-vinyl)3β,17β-androst-5-ene diol;
19-nor-10β-(1-ethyl-vinyl)-3β,17β-androst-5-ene diol;
19-nor-10β-(cis-2-ethyl-vinyl)-3β,17β-androst-5-ene diol;
19-nor-10β-(trans-2-ethyl-vinyl)-3β,17β-androst-5-ene diol;
19-nor-10β-(1-chloro-vinyl)-3β,17β-androst-5-ene diol;
19-nor-10β-(cis-2-chloro-vinyl)-3β,17β-androst-5-ene diol;
19-nor-10β-(trans-2-chloro-vinyl)-3β,17β-androst-5-ene diol;
19-nor-10β-(1-fluoro-vinyl)-3β,17β-androst-5-ene diol;
19-nor-10β-(cis-2-fluoro-vinyl)-3β,17β-androst-5-ene diol;
19-nor-10β-(trans-2-fluoro-vinyl)-3β,17β-androst-5-ene diol;
19-nor-10β-(2,2-difluoro-vinyl)-3β,17β-androst-5-ene diol;
19-nor-10β-(trifluorovinyl)-3β,17β-androst-5-ene diol;
17α-ethynyl-19-nor-10β-trifluorovinyl-3β,17β-androst-5-ene diol; or a pharmaceutically acceptable salt or stereoisomer thereof.

7. A pharmaceutical composition comprising a compound of the formula 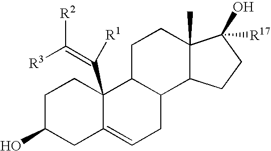
wherein R1 is hydrogen, halo, C₍₁₋₃₎ alkyl, CO₂C₍₁₋₃₎alkyl, or cyano;
R2 is hydrogen, halo, C₍₁₋₃₎ alkyl, CO₂C₍₁₋₃₎alkyl, or cyano;
R3 is hydrogen, halo, C₍₁₋₃₎ alkyl, CO₂C₍₁₋₃₎alkyl, or cyano;
R17 is hydrogen, C₍₁₋₅₎ alkyl, C₍₁₋₅₎ acyl, C₍₂₋₅₎ alkenyl, or C₍₂₋₅₎ alkynyl;
or a pharmaceutically acceptable salt or stereoisomer thereof;
and another agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; or a selective serotonin reuptake inhibitor; an aromatase inhibitor; or a pharmaceutically acceptable salt or mixture thereof.

8. The method of claim 1 further comprising another agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; an osteoblast anabolic agent; calcitonin; Vitamin D; a synthetic Vitamin D analogue; or a selective serotonin reuptake inhibitor; an aromatase inhibitor; or a pharmaceutically acceptable salt or mixture thereof.

9. A compound of formula II 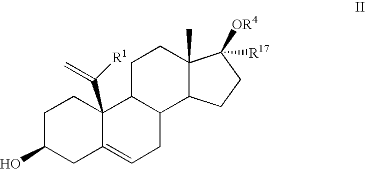
wherein R¹ is hydrogen, halo, C₍₁₋₃₎ alkyl, or cyano;
R⁴ is hydrogen, or C₍₁₋₃₎ alkyl;
R¹⁷ is hydrogen, C₍1-5) alkyl, C₍₂₋₅₎ alkenyl, or C₍₂₋₅₎ alkynyl;
with the proviso that R¹ and R⁴ are not both hydrogen or a pharmaceutically acceptable salt or stereoisomer thereof.

10. A pharmaceutical composition comprising a compound of claim 9.
